(19) United States
(12) Patent Application Publication
(10) Pub. No.: US 2013/0209592 A1
(43) Pub. Date: Aug. 15, 2013

(54) Plant Extract Based Compositions and Methods For Treating Chronic Wounds

(71) Applicant: Steven C. Denstman, Sarasota, FL (US)

(72) Inventor: Steven C. Denstman, Sarasota, FL (US)

(21) Appl. No.: 13/371,989

(22) Filed: Feb. 13, 2012

(51) Int. Cl. A61K 36/899; A61P 31/04

(52) U.S. Cl. 424/750

(57) ABSTRACT

A therapeutic healing compound is provided. The therapeutic healing compound includes a sugarcane plant extract and a gelling agent that includes at least one of xanthan gum and hydroxyethyl cellulose. The compound may also include collagen. The therapeutic healing compound may be used for treating wounds, burns and cosmetic enhancement, such as an exfoliate for anti-aging.

### BACKGROUND OF THE INVENTION

The field of the invention relates generally to compositions for treating chronic wounds and more particularly, to plant extract based compositions and methods for treating chronic wounds.

The prevalence of chronic wounds is increasing in parallel with an aging population. The cost of treating chronic wounds is rapidly increasing. This upward trend places a strain on the health care system and negatively impacts morbidity and mortality rates. Patients suffering with chronic wounds may experience a compromised quality of life, restrictive lifestyle changes and excess financial burdens.

The temporal aspects of wound healing are important. The longer a wound remains open-susceptible, the risk of bacterial super-infection, systematic bacterial infection, necrosis, gangrene, and the potential risk for amputation of an extremity, increases dramatically. The physiology of wound healing is sometimes misunderstood. The wound healing process is complex and proceeds in phases that can take from weeks to months to complete. The rate of this process can be influenced by a number of factors that include age, overall health, nutritional status and underlying co-morbidity.

Typically, the majority of chronic wounds occur in people over age 65. The most common causes of wounds in this population are trauma, decubitus ulcers (bedsores) caused by generalized debility with a sedentary life style and the inability to easily move, diabetic ulcers caused by poor circulation and nerve damage, peripheral vascular disease and/or poor arterial circulation, and venous stasis with chronic edema due to incompetent vein valves.

The standard approach to treating wounds involves cleaning and debriding, treating infection, addressing circulatory issues, and dressing the wound. A number of adjunctive treatments have become available with the intent of expediting the wound healing process. Most of the commercially available wound healing agents are expensive and some are of questionable utility. The majority of these products are based on a collagen matrix foundation that provides the strata for fibroblast growth and repair of the injured tissue. Antibacterial compounds like silver, and growth factors that fibroblast growth theoretically hastening the healing process are often added to these products.

It would be advantageous to provide an effective, cost efficient wound healing product that may save money for patients, and improve the health and quality of life of a significant portion of the world population.

### BRIEF DESCRIPTION OF THE INVENTION

In one aspect, a therapeutic healing compound is provided. The therapeutic healing compound includes a sugarcane plant extract and a gelling agent that includes at least one of xanthan gum and hydroxyethyl cellulose. The compound may also include collagen.

In another aspect, a method of healing a wound or burn is provided. The method includes applying a therapeutic healing compound to a wound and covering the wound and therapeutic healing compound with a dressing. The therapeutic healing compound includes a sugarcane plant extract, a gelling agent that includes at least one of xanthan gum and hydroxyethyl cellulose. The compound may also include collagen.

In another aspect, a therapeutic healing compound is provided. The therapeutic healing compound includes about 35 percent to about 75 percent by weight of a sugarcane plant extract, about 20 percent to about 60 percent of water, a gelling agent that includes at least one of xanthan gum and hydroxyethyl cellulose, and collagen.

### BRIEF DESCRIPTION OF THE DRAWINGS

FIG. 1 is a graph of the wound size (percent of the original wound) over time for responding patients.

FIG. 2 is a graph of the wound size (percent of the original wound) over time for non-responding patients.

FIG. 3 is a schematic illustration of the experimental design of a deep partial thickness wound study.

FIG. 4 is a bar graph showing the percent of re-epithelialization for each treatment on day 6.

FIG. 5 is a bar graph showing the percent of re-epithelialization for each treatment on day 7.

FIG. 6 is a bar graph showing the percent of re-epithelialization for each treatment on day 8.

FIG. 7 is a bar graph showing the percent of re-epithelialization for each treatment on day 9.

FIG. 8 is a bar graph showing the percent of re-epithelialization for each treatment on day 10.

FIG. 9 is a graph showing the percentage of wounds completely re-epithelialized on each assessment day.

FIG. 10 is a bar graph showing the percentage of wounds completely re-epithelialized on each assessment day for the wound healing compound embodiment that includes hydroxyethyl cellulose.

FIG. 11 is a graph showing the percentage of wounds completely re-epithelialized on each assessment day for the wound healing compound embodiment that includes hydroxyethyl cellulose.

FIG. 12 is a bar graph showing the percentage of wounds completely re-epithelialized on each assessment day for the wound healing compound embodiment that includes xanthan gum.

FIG. 13 is a graph showing the percentage of wounds completely re-epithelialized on each assessment day for the wound healing compound embodiment that includes xanthan gum.

FIG. 14 is a bar graph showing the percentage of wounds epithelialized at day 3 and day 5.

FIG. 15 is a bar graph showing the epithelial thickness at day 3 and day 5.

FIG. 16 is a schematic illustration of the experimental design of an anti microbial study.

FIG. 17 is a bar graph of bacterial counts after 48 hours of treatment.

FIG. 18 is a bar graph of in vitro anti-microbial activity against Pseudomonas aeruginosa.

FIG. 19 is a bar graph of in vitro anti-microbial activity against Methicillin resistant staphylococcus aureus.

### DETAILED DESCRIPTION OF THE INVENTION

Plant extract based treatment compositions and methods for treating chronic wounds and burns are described below. The plant extract may be an extract from sugarcane. The sugarcane extract may be filtered and/or boiled at high temperatures until its concentration permits the crystallization of the extract. The crystallized extract typically includes sugars (e.g., sucrose, glucose, and fructose), and may also include vitamins (e.g., A, B complex, C, D, and E), and minerals (e.g., potassium, calcium, phosphorus, magnesium, iron, copper, zinc, and manganese). Sucrose is the principle constituent of panela with a content typically varying from between about 75% to about 85% by dry weight. Glucose and fructose are typically present between about 6% to about 15% by dry weight. The plant extract based treatment compositions facilitates quicker initiation of the wound healing process than known treatment compounds. Also, the plant extract based treatment compositions stimulates wounds to complete healing faster than known treatment compounds. In addition, the plant extract based treatment compositions possess both In Vitro and In Vivo antimicrobial activity against both Pseudomonas aeruginosa and Methicillin resistant staphylococcus aureus (MRSA). The description below focuses on the treatment of wounds and burns; however, the treatment compounds may also be utilized in cosmetic enhancements, for example, as an exfoliate for anti-aging properties.

In an exemplary embodiment, a plant extract based treatment composition includes a plant extract and a hydroxyethyl cellulose carrier. The hydroxyethyl cellulose may act as a gelling agent to form a gelled treatment composition. The plant extract may be a sugarcane extract that has been crystallized by a boiling process. The sugarcane extract is in the form of a juice that is heated to about 99° C. to evaporate most of the liquid, and then heated to about 130° C. to crystallize the sugarcane extract. The treatment composition also may include water, potassium sorbate, collagen hydrolysate powder, ascorbic acid powder, vitamin E acetate, and polysorbate 80. In one embodiment, the treatment composition includes about 70% to about 75% by wt. of sugarcane extract, about 20% to about 25% of water, about 1.0% to about 2.0% of hydroxyethyl cellulose, about 0.2% to about 0.3% of potassium sorbate, about 0.15% to about 0.25% of collagen hydrolysate, about 0.5% to about 1.5% ascorbic acid, about 0.5% to about 1.5% of vitamin E acetate, and about 0.05% to about 0.15% polysorbate 80. In addition, sodium hydroxide may be added to adjust the pH to about 6.

In another embodiment, the treatment composition includes sugarcane extract and a xanthan gum carrier. The treatment composition also may include water, potassium sorbate, collagen hydrolysate powder, ascorbic acid powder, vitamin E acetate, polysorbate 80, and glycerine. Particularly, the treatment composition includes about 35% to about 45% by wt. of sugarcane extract, about 50% to about 60% of water, about 0.2% to about 0.6% of xanthan gum, about 0.1% to about 0.2% of potassium sorbate, about 0.05% to about 0.15% of collagen hydrolysate, about 0.5% to about 1.0% ascorbic acid, about 0.5% to about 1.0% of vitamin E acetate, about 0.03% to about 0.1% polysorbate 80, and about 1.5% to about 3.5% glycerine. In addition, sodium hydroxide may be added to adjust the pH to about 6.

In another embodiment, the treatment composition includes about 35% to about 75% by wt. of sugarcane extract, about 20% to about 60% of water, about 0.2% to about 2.0% of a gelling agent, about 0.1% to about 0.3% of potassium sorbate, about 0.05% to about 0.25% of collagen hydrolysate, about 0.5% to about 1.5% ascorbic acid, about 0.5% to about 1.5% of vitamin E acetate, about 0.03% to about 0.15% polysorbate 80, and about 0% to about 3% glycerin. In addition, sodium hydroxide may be added to adjust the pH to about 6. The gelling agent may be hydroxyethyl cellulose or xanthan gum.

A method of healing wounds and burns on a patient may include cleaning the wound or burns and/or debriding the wound. The treatment composition is then applied to the wound or burn. The treatment composition may be applied multiple times on a regular basis (e.g., daily, hourly, etc.). An effective amount of the therapeutic treatment composition is applied to wounds and burns on a patient, including chronic wounds. By effective amount is meant to be an amount of the treatment composition that results in measurable amelioration of at least one symptom or parameter of the wound or burn. The effective amount for treating the different wounds and burns can be determined by, for example, establishing a matrix of dosages and frequencies of application and comparing a group of subjects to each point in the matrix. Typically, a dressing is also applied to the treatment composition. Any known dressing may be used, for example, a saline dressing, a plastic film, gauze, and the like. In addition, the treatment composition may be used as a cosmetic enhancement, for example, as an exfoliate for anti-aging of skin.

### EXAMPLES

Four test examples are described below. Example 1 was a human pilot study that studied wound healing properties of one embodiment of the wound healing compound described above. Example 2 was a study that examined the effect of the wound healing compound on the healing of deep partial thickness wounds using a porcine model. Two similar formulations using a hydroxyethyl cellulose gel or a xantham gum based carrier were tested. Example 3 was a histological analysis of Example 2 to determine the percent re-epithelialization of the technique described in Example 2. Example 4 was an anti-microbial study to determine the effect of the wound healing compound on Pseudomonas aeruginosa using a deep partial thickness porcine wound model.

### Example I

A human pilot study was conducted at a well known hospital based wound center in the United States. The patient population consisted of both males and females who were diagnosed with a moderate to severe wound. All of the patients had a history of a coexisting co-morbid condition that is usually associated with compromised wound healing (Diabetes Mellitus, Peripheral Vascular Disease, Venous Insufficiency), as well as a personal history of compromised wound healing. Twenty four adult patients were enrolled in the study. The study patients were formally consented.

The wound healing compound used in this example was formulated by the following ingredients shown in Table 1.

TABLE 1

| Ingredients | Quantity Used | |
|---|---|---|
| Sugarcane Block | 75 | GM |
| Water (Purified) | 24.05 | GM |
| Hydroxyethyl Cellulose NF (5000 CPS) | 1.5 | GM |
| Potassium Sorbate Powder | 0.25 | GM |
| Collagen Hydrolysate Powder | 0.2 | GM |
| Ascorbic Acid Powder | 1 | GM |
| Vitamin E Acetate (DL) 1 MG/ML Liquid | 1 | ML |
| Polysorbate 80 Liquid | 3 | Drops |
| Sodium Hydroxide (10% Solution) | | |

The ingredients were mixed by weighing the potassium sorbate in a beaker and weighing water in the same beaker with the potassium sorbate. Collagen hydrolysate and ascorbic acid were then added to the beaker. The pH was adjusted to about 6 with a 10% solution of sodium hydroxide. Then the vitamin E acetate was triturated with polysorbate 80 and added to the beaker. The sugarcane block was triturated with a portion of the liquid in the beaker to form a paste. Then the remainder of the liquid in the beaker was added to the paste and spun until dissolved. Next the hydroxyethyl cellulose was added to the composition and spun until gelled. The composition was dispensed into an empty jar and stored at room temperature.

The subject wounds were all cleaned and debrided utilizing standard medical techniques. Patients were instructed to apply the healing compound, described above, to open wounds on a daily basis and to cover wounds with a moist saline dressing. Patients were followed on a weekly basis with wound measurements and photographs until such time that the wound was healed or exhibited signs of progression.

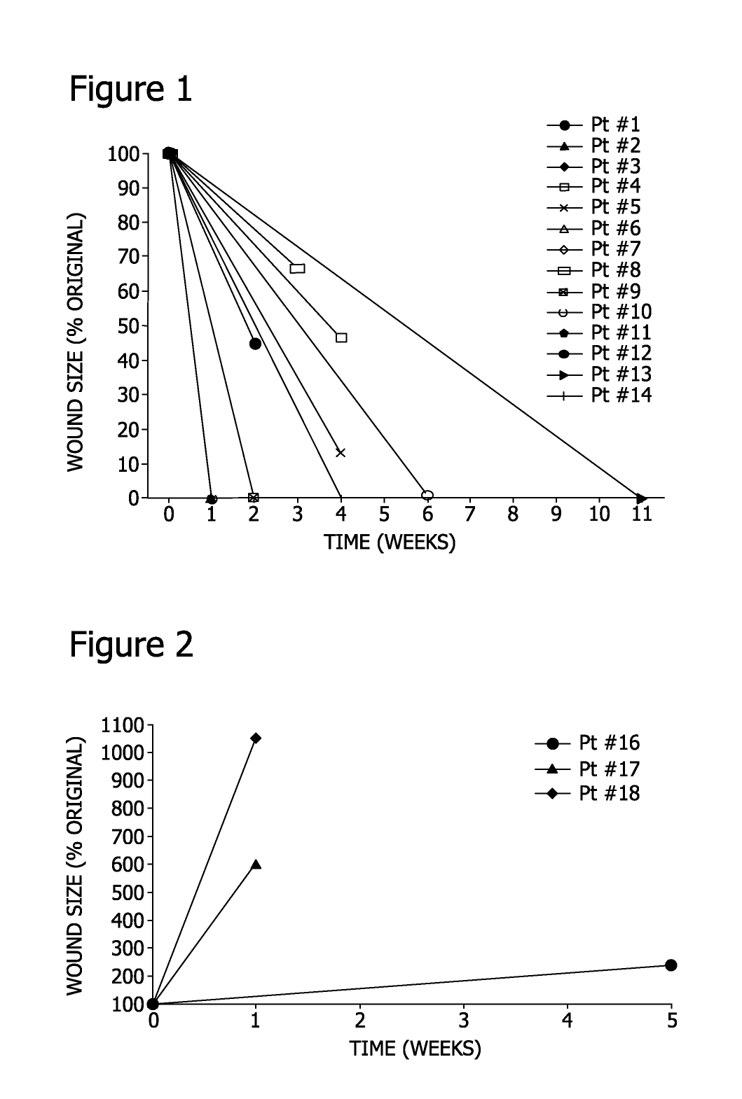

The healing compound was well tolerated as none of the patients complained of toxicity or any other untoward side effects. Eighteen patients were deemed responders and three subjects were non-responders. One patient expired related to a longstanding cardiac issue and two patients were lost to follow-up. The wound healing kinetics are shown for a representative subset of the patients from both groups in the graphs shown in FIGS. 1 and 2. FIG. 1 shows the wound healing kinetics of the responder patients, and FIG. 2 shows wound healing kinetics of the non-responder patients.

This simple pilot study shows that the wound healing compound is a cost effective natural product with excellent activity as an adjunctive wound healing agent in this population of patients who can be categorized as “poor healers”.

### Example II

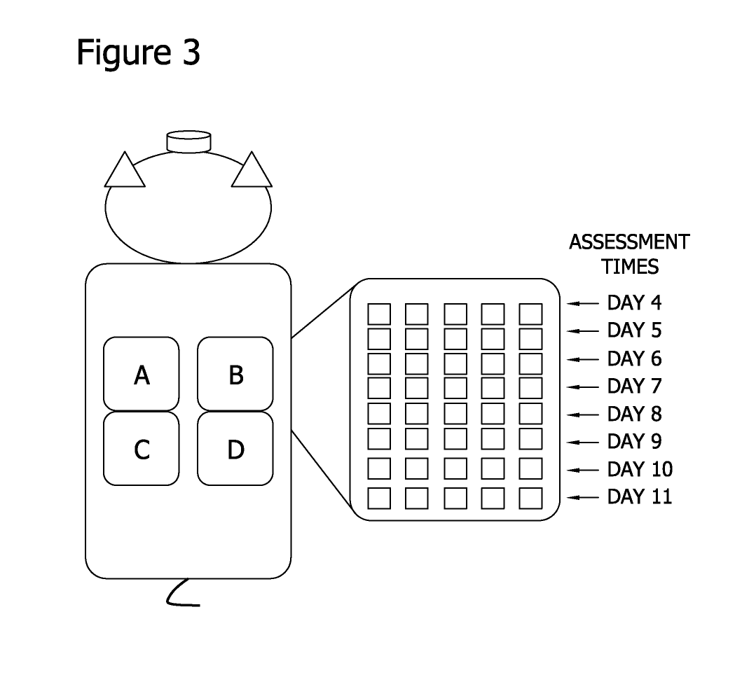

This example study examined the effect of the healing compound described in Example I, on the healing of deep partial thickness wounds using a well established porcine model. One hundred and sixty (160) rectangular wounds measuring 10 mm×7 mm×0.5 mm deep were made in the paravertebral and thoracic area with a specialized electrokeratome fitted with a 7 mm blade. The wounds were separated from one another by 15 mm of unwounded skin. As shown in FIG. 3, the wounds were divided into four treatments groups (A, B, C, D) of 40 wounds in each group. The wounds of each treatment group were then treated. Treatment Group A was treated with a cellulose gel base compound that included hydroxyethyl cellulose, potassium sorbate and water, treatment Group B was treated with a base plant extract compound that included a sugarcane extract, hydroxyethyl cellulose, potassium sorbate, polysorbate, and water, treatment Group C was treated with the wound healing compound described above in Example I, and treatment Group D was an untreated control group. The treatment compounds were covered with a polyurethane film dressing.

The number of wounds healed (completely epithelialized) was divided by the total number of wounds sampled per day for the corresponding treatment group and multiplied by 100 to obtain the percentage of healed wounds. None of the groups had any wounds completely healed on day 4 and/or day 5 after wounding (day 0).

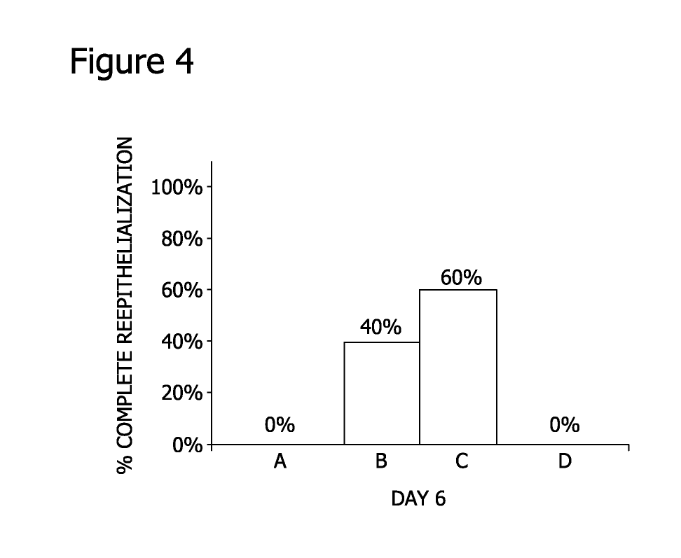

As shown in FIG. 4, on day 6, 60% of the wounds treated with the healing compound described above in Example I, in Group C were completely re-epithelialized. In Group B, 40% of the wounds treated with the base plant extract completely re-epithelialized. None of the wounds in cellulose gel base Group A and untreated Group D were re-epithelialized.

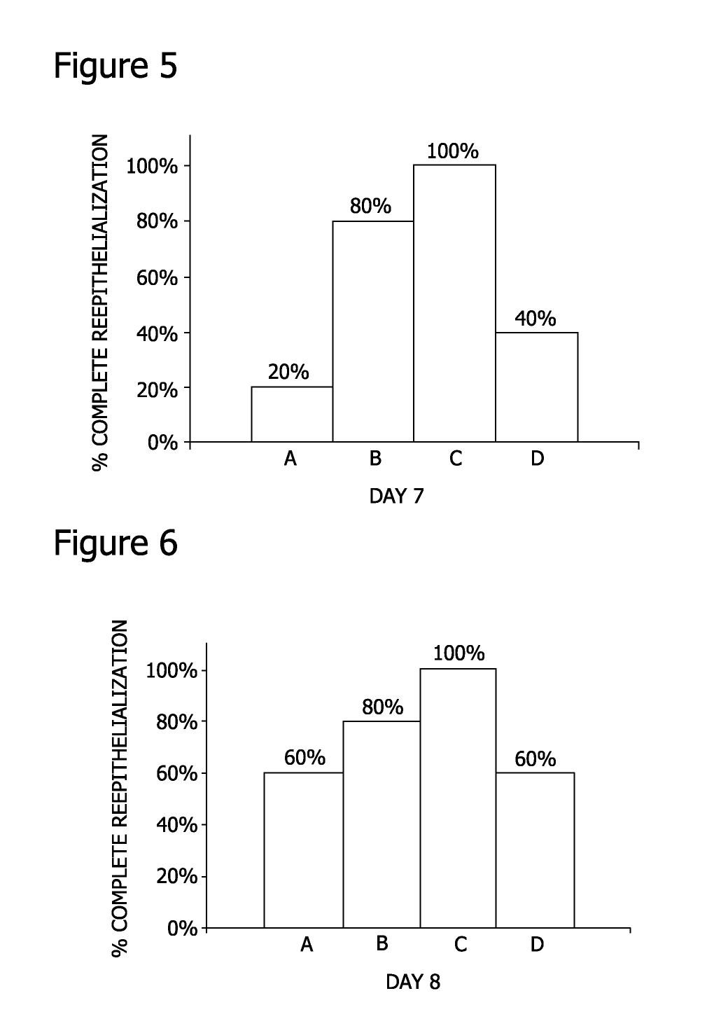

As shown in FIG. 5, on day 7, 100% of wounds treated with the wound healing compound described above in Example I, in Group C were completely re-epithelialized. In Group B and Group D had 80% and 40% of the wounds completely re-epithelialized, respectively. In Group A, 20% of wounds were completely re-epithelialized.

As shown in FIG. 6, on day 8, 100% of wounds treated with the wound healing compound described above in Example I, in Group C were completely re-epithelialized. In Group B, 80% of the wounds were completely re-epithelialized. In Group A and Group D 60% of wounds were completely re-epithelialized.

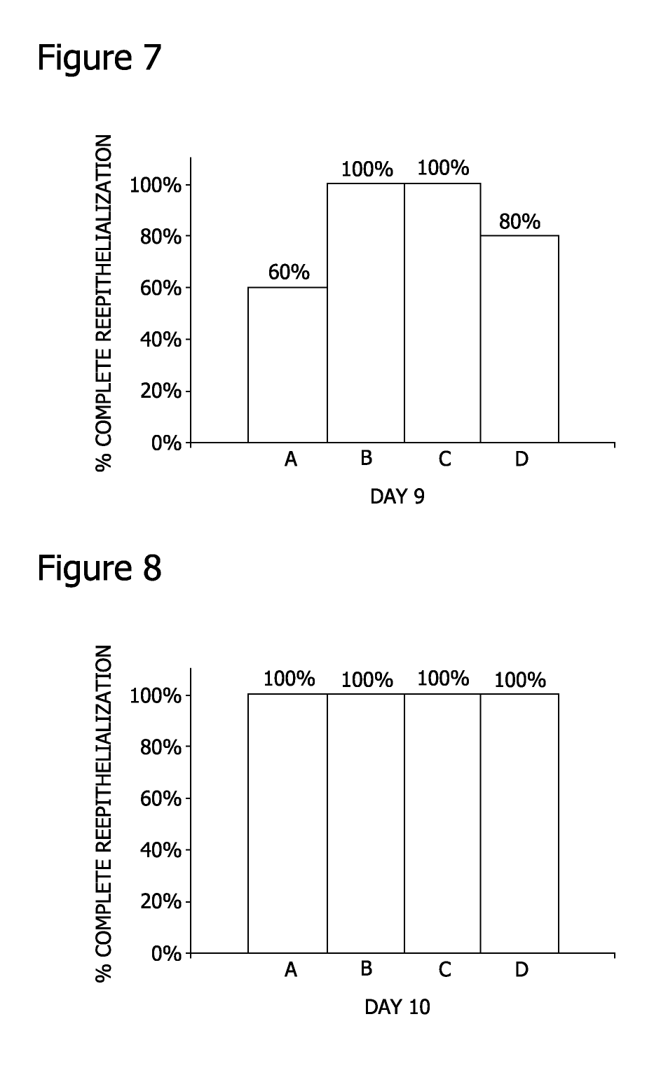

As shown in FIG. 7, on day 9, 100% of wounds treated with the wound healing compound described above in Example I, and the wounds treated with base plant extract of Group B were completely re-epithelialized. The untreated wounds of Group D were 80% completely re-epithelialized while wounds treated with cellulose gel case of Group A were 60% completely re-epithelialized.

As shown in FIG. 8, on day 10, all treatment groups A, B, and C, including the untreated control Group D were 100% re-epithelialized.

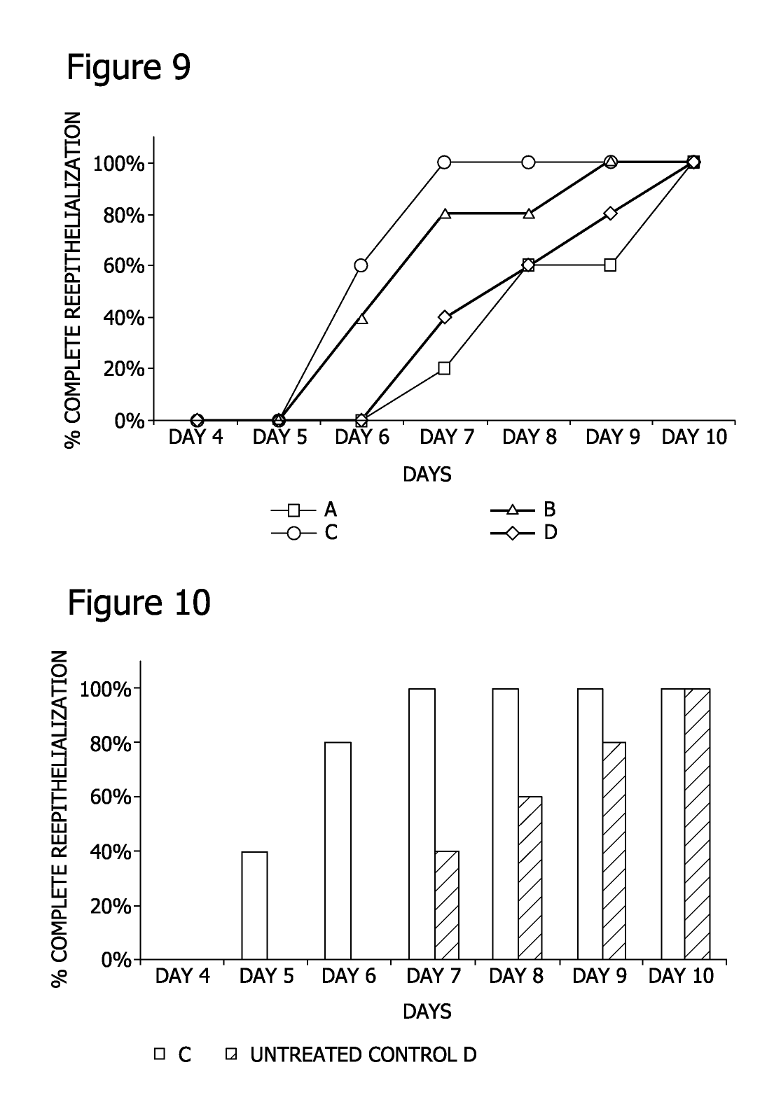

As shown in FIG. 9, the wound healing compound of Group C re-epithelialized more rapidly than base plant extract, cellulose gel base and untreated wounds. Wounds treated with the wound healing compound of Group C initiated complete healing four days (day 7) before the untreated controls (day 10). This study suggests that the healing compound of Group C was effective in increasing the re-epithelialization rate of deep partial thickness wounds.

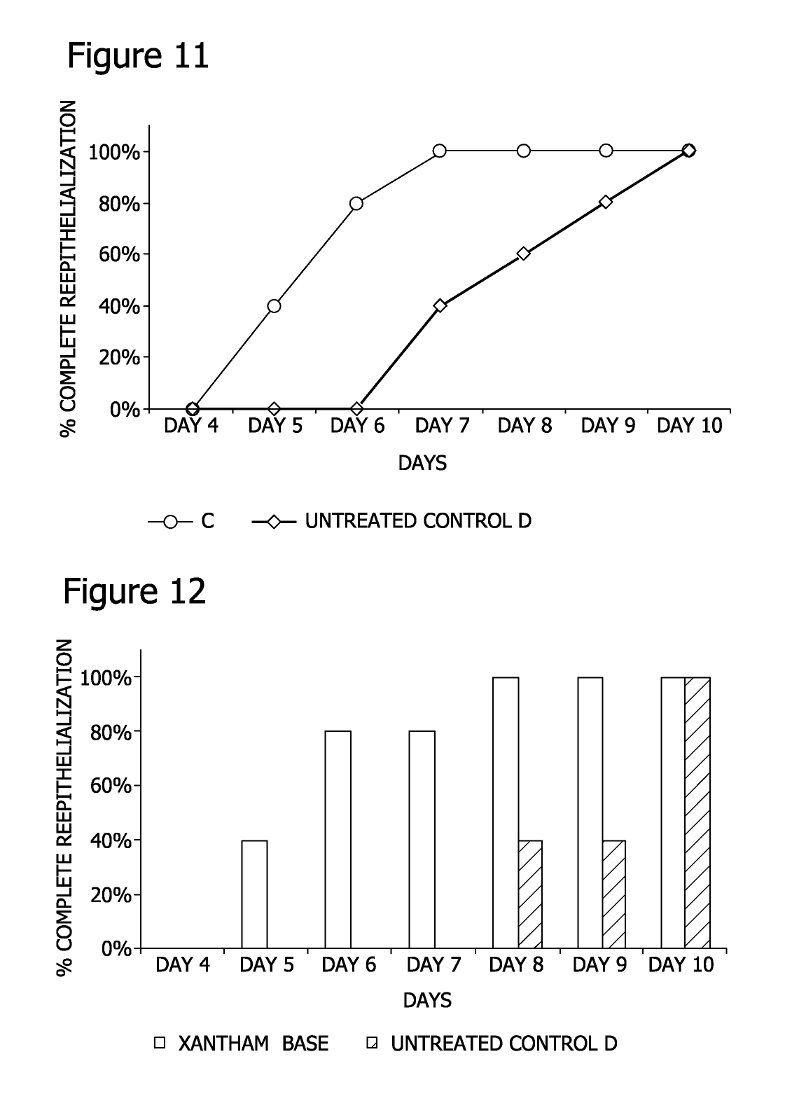

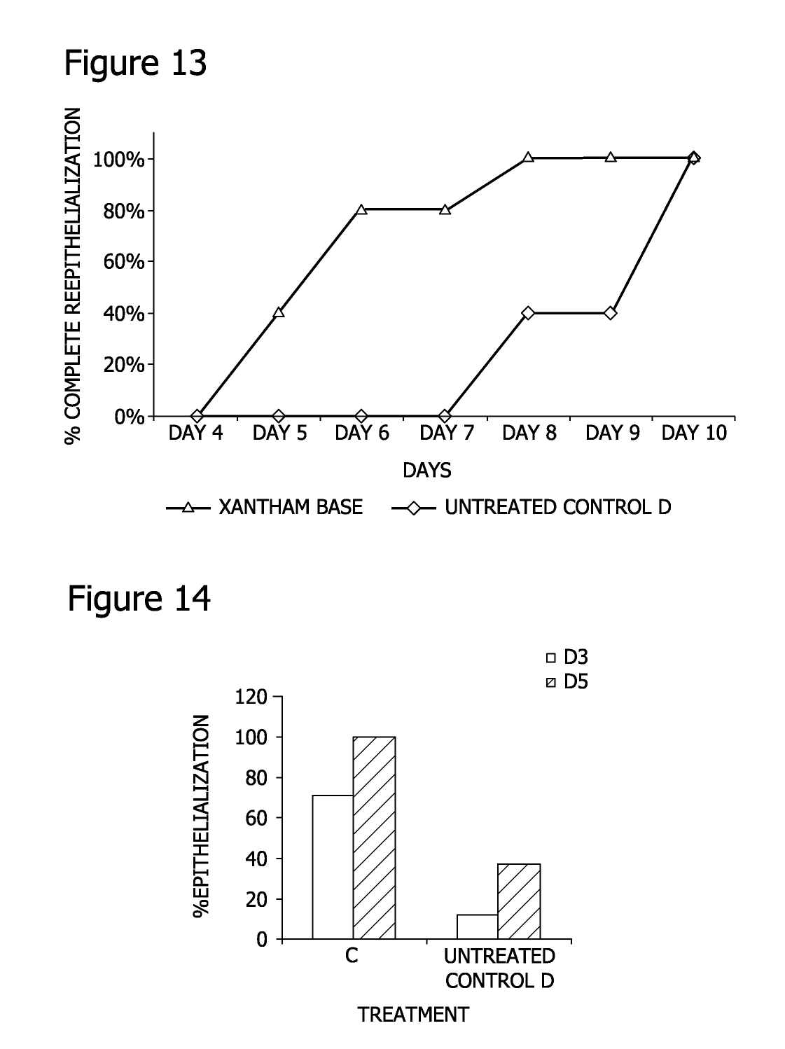

The results were duplicated on two additional pigs in two tests that compared the healing compound with an untreated control. The first duplicate test was conducted with the first healing compound described above in Example I. The second duplicate test was conducted with a second healing compound having a formulation that included a xantham gum based carrier which showed a similar reproducible activity. The second compound was formulated by the following ingredients shown in Table 2. FIGS. 10 and 11 show the results of the first duplicate test, and FIGS. 12 and 13 show the results of the second duplicate test.

TABLE 2

| Ingredients | Quantity Used | |
|---|---|---|
| Sugarcane Block | 75 | GM |
| Water (Purified) | 100 | GM |
| Potassium Sorbate Powder | 0.25 | GM |
| Collagen Hydrolysate Powder | 0.2 | GM |
| Ascorbic Acid Powder | 1 | GM |
| Vitamin E Acetate (DL) 1/MG/ML Liquid | 1 | ML |
| Polysorbate 80 Liquid | 3 | Drops |
| Sodium Hydroxide (10% Solution) | | |
| Xanthan Gum Powder | 0.75 | GM |
| Glycerin Liquid | 5 | ML |

The ingredients were mixed by weighing the potassium sorbate in a beaker and weighing water in the same beaker with the potassium sorbate. Collagen hydrolysate and ascorbic acid were then added to the beaker. The pH was adjusted to about 6 with a 10% solution of sodium hydroxide. Then the vitamin E acetate was triturated with polysorbate 80 and added to the beaker. The sugarcane block was triturated with a portion of the liquid in the beaker to form a paste. Then the remainder of the liquid in the beaker was added to the paste and spun until dissolved. Next the xanthan gum was triturated with glycerin to a paste, and then added to the composition to form a paste. The composition was dispensed into an empty jar and stored at room temperature.

### Example III

To further evaluate the activity of the second wound healing compound described in Example II, wounds from the pig in the second duplicate test described above were evaluated by histological analysis to determine the percent re-epithelialization. Deep partial thickness wounds were characterized by removal of the entire epidermis and only a portion of the dermis. In this porcine wound healing model healing occurred by migration of epithelial cells from the wound edge as well as from edge of the epidermal appendages (e.g. hair follicles).

Histologic analysis was performed blindly in duplicate by a Pathologist without knowing whether treated wounds or untreated controls were being examined. The analysis showed increased re-epithelialization with the second wound healing compound described above in Example II versus untreated controls at both day 3 and day 5 of analysis.

The percent of re-epithelialization represents the percent of the wound area covered by newly formed epithelium, or the epidermis with one or more layers of keratinocytes which is considered a good index for the speed of keratinocyte migration. The second wound healing compound resulted in much faster re-epithelialization on both day 3 and day 5 when compared to untreated controls as shown in FIG. 14.

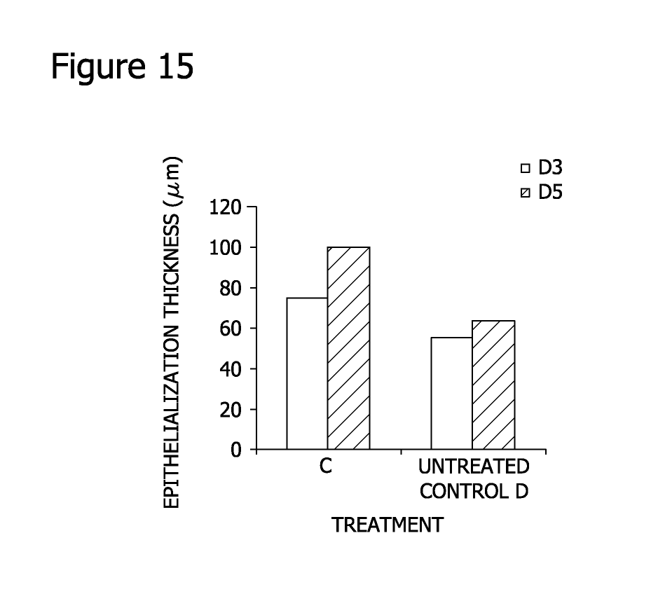

The epithelial thickness is a measure of an average thickness at five points of newly formed epithelium. Epithelial thickness reflects the process of keratinocyte proliferation, differentiation and epidermal maturation. Compared with the untreated control, thicker epithelia were observed in the second wound healing compound treatment group as shown in FIG. 15.

### Example IV

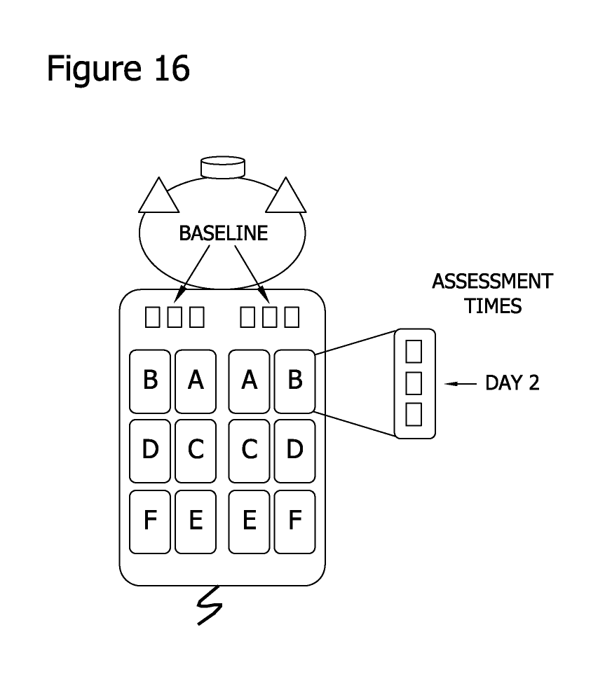

An anti-microbial study was conducted to determine the effect of the wound treatment compound described in Example I, on Pseudomonas aeruginosa (ATCC27312), using a deep partial thickness porcine wound model. Forty two rectangular wounds measuring 10 mm×7 mm×0.5 mm deep were made in the paravertebral and thoracic area with a specialized electrokeratome fitted with a 7 mm blade. The wounds were separated from one another by 15 mm of unwounded skin and individually dressed. Three wounds were randomly assigned to each treatment group and they were inoculated. As shown in FIG. 16, the wounds were divided into six treatments groups (A, B, C, D, E, F) of 3 wounds in each group. The wounds of each treatment group were then treated. Treatment Group A was treated with a cellulose gel base compound that included hydroxyethyl cellulose, potassium sorbate and water, treatment Group B was treated with a base plant extract compound that included a sugarcane extract, hydroxyethyl cellulose, potassium sorbate, polysorbate, and water, treatment Group C was treated with the wound healing compound described above in Example I that includes hydroxyethyl cellulose, treatment Group D was treated with a compound having a honey base, treatment Group E was treated with a compound having a positive control anti-microbial (mupirocin for MRSA and silver sulfadiazine for Pseudomonas aeruginosa), and treatment Group F was an untreated control group. The treatment compounds were covered with a polyurethane film dressing.

Immediately after wounding, the wounds were inoculated with the appropriate bacterial strain. All wounds were covered, individually, with a polyurethane film dressing (Tegaderm; 3M, St. Paul, Minn.). Polyurethane film dressings were secured along the edges using surgical tape. All dressings were covered and secured by wrapping the animal with self-adherent elastic bandages (Coban; 3M, St. Paul, Minn.). The dressings were left in place for 24 hours to allow formation of a bacterial biofilm in the wounds. After 24 hours, the dressings were removed. Three of the wounds were recovered for baseline bacterial counts. The remaining wounds were divided into six groups of three wounds each and treated once daily with the appropriate treatment groups A-E. Topical formulations were also covered with a polyurethane film dressing individually to prevent any cross contamination.

After the incubation period, colonies were counted, the data was tabulated and the Log of colony forming units/ml (Log CFU/ml) for Pseudomonas aeruginosa (PA) determined. The arithmetic mean of the Log (CFU/ml) and standard deviation were calculated for each treatment.

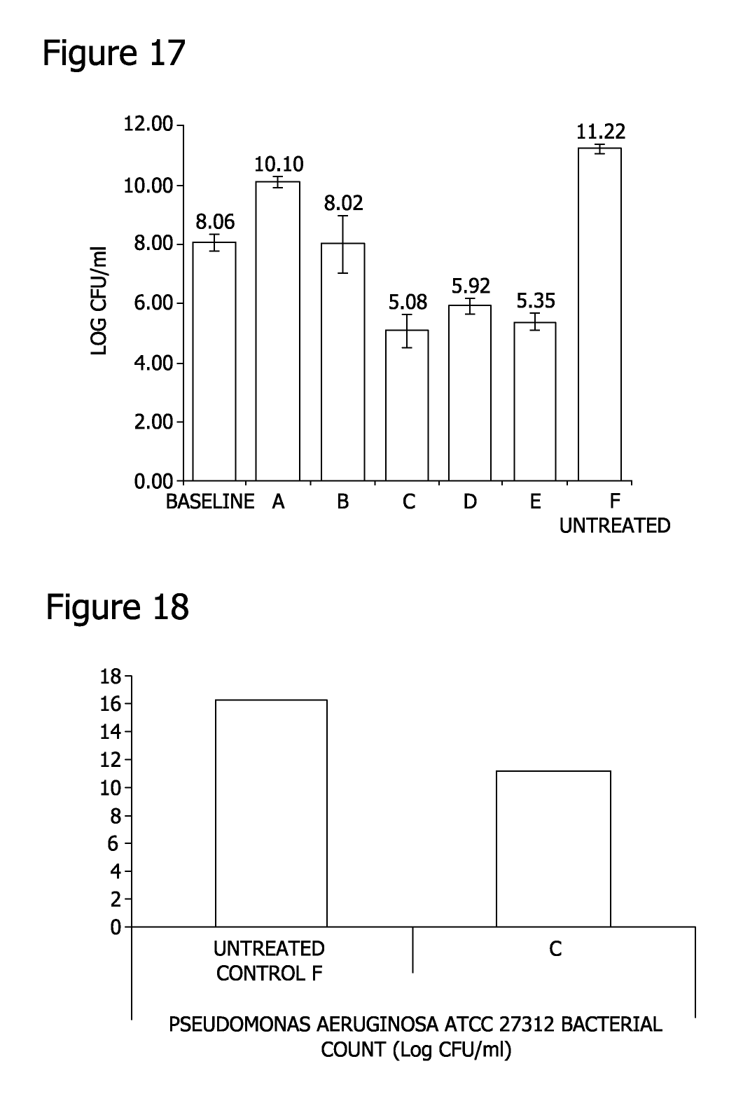

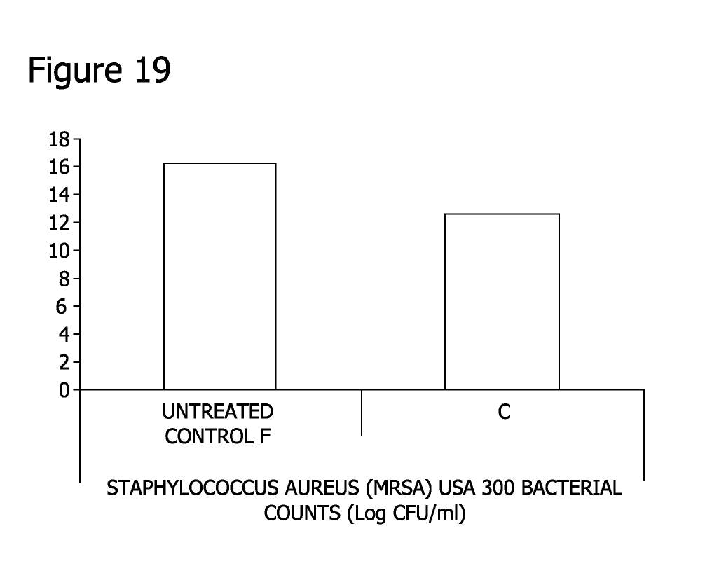

Baseline wounds (prior to treatment) contained 8.06±0.28 Log CFU/ml of PA after 24 hours biofilm formation. Wounds treated with the wound treatment compound described in Example I had the lowest PA counts (5.08±0.58 Log CFU/ml) compared to other treatments as shown in FIG. 17. The lower bacterial count in wounds treated with the wound treatment compound described in Example I was followed by Silver Sulfadiazine (5.35±0.35) and the honey based compound (5.92±0.28) Log CFU/ml. Base plant extract and cellulose gel base had (8.02±0.99 and 10.10±0.17 Log CFU/ml, respectively) of PA recovered from wounds. Wounds in the untreated group resulted in the highest Log CFU/ml (11.22±0.17) of PA.

As shown in FIGS. 17 and 18, the wound treatment compound described in Example I, demonstrates In Vitro antimicrobial activity against Pseudomonas Aeruginosa and Methicillin Resistant Staphylococcus Aureus (MRSA) when studied using a simple In Vitro bacterial colony forming assay.

In general, reducing Pseudomonas aeruginosa populations in inoculated wounds carries important clinical implications for wound treatment and the prevention of infections. It is possible that if treatments were applied twice daily a larger increase in bacterial reduction may have been observed.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A therapeutic healing compound having an anti-bacterial property, said therapeutic healing compound comprising:
a sugarcane plant extract;
a gelling agent comprising at least one of xanthan gum and hydroxyethyl cellulose; and
collagen.

2. The therapeutic healing compound in accordance with claim 1 wherein said healing compound further comprises water.

3. The therapeutic healing compound in accordance with claim 1 wherein said compound further comprising at least one of potassium sorbate, ascorbic acid, vitamin E, polysorbate, and sodium hydroxide.

4. The therapeutic healing compound in accordance with claim 1 wherein said gelling agent comprises a cellulose gel.

5. The therapeutic healing compound in accordance with claim 1 wherein said gelling agent comprises a xanthan gum based carrier.

6. The therapeutic healing compound in accordance with claim 5 wherein said compound further comprises glycerin.

7. A method of healing a wound or a burn, the method comprising:
applying a therapeutic healing compound to a wound or burn; and
covering the wound or burn and therapeutic wound healing compound with a dressing;
the therapeutic healing compound having an anti-bacterial property, the therapeutic healing compound comprising:
a sugarcane plant extract;
a gelling agent comprising at least one of xanthan gum and hydroxyethyl cellulose; and
collagen.

8. The method of healing a wound or a burn in accordance with claim 7 further comprising applying the therapeutic healing compound to the wound or burn multiple times.

9. The method of healing a wound or a burn in accordance with claim 8 further comprising covering the wound or burn, and the therapeutic healing compound with a dressing each time the therapeutic healing compound is applied to the wound or burn.

10. The method of healing a wound or a burn in accordance with claim 7 wherein applying a therapeutic healing compound comprises applying a healing compound that further comprises water.

11. The method of healing a wound or a burn in accordance with claim 7 wherein applying a therapeutic healing compound comprises applying a healing compound that further comprises at least one of potassium sorbate, ascorbic acid, vitamin E, polysorbate, and sodium hydroxide.

12. The method of healing a wound or a burn in accordance with claim 7 wherein the thickener gelling agent comprises a cellulose gel.

13. The method of healing a wound or a burn in accordance with claim 7 wherein the gelling agent comprises a xanthan gum based carrier.

14. The method of healing a wound or a burn in accordance with claim 13 wherein applying a therapeutic healing compound comprises applying a healing compound that further comprises glycerin.

15. A therapeutic healing compound having an anti-bacterial property, said therapeutic healing compound comprising:
about 35 percent to about 75 percent by weight of a sugarcane plant extract;
about 20 percent to about 60 percent of water;
about 0.2 percent to about 2.0 percent of a gelling agent comprising at least one of xanthan gum and hydroxyethyl cellulose; and
about 0.05 percent to about 0.15 percent of collagen hydrolysate.

16. The therapeutic healing compound in accordance with claim 15 wherein said compound further comprises at least one of potassium sorbate, ascorbic acid, vitamin E, polysorbate, and sodium hydroxide.

17. The therapeutic healing compound in accordance with claim 15 wherein said gelling agent comprises a cellulose gel comprising hydroxyethyl cellulose.

18. The therapeutic healing compound in accordance with claim 15 wherein said gelling agent comprises a xanthan gum based carrier.

19. The therapeutic healing compound in accordance with claim 18 wherein said compound further comprises glycerin.
